(19) United States
(12) Patent Application Publication
(10) Pub. No.: US 2013/0217002 A1
(43) Pub. Date: Aug. 22, 2013

(54) HIV-1 IGG3 RESPONSE IN ACUTE HIV-1

(71) Applicants: Georgia D. Tomaras, Durham, NC (US); Nicole L. Yates, Durham, NC (US); Myron S. Cohen, Chapel Hill, NC (US); Barton F. Haynes, Durham, NC (US)

(72) Inventors: Georgia D. Tomaras, Durham, NC (US); Nicole L. Yates, Durham, NC (US); Myron S. Cohen, Chapel Hill, NC (US); Barton F. Haynes, Durham, NC (US)

(73) Assignees: DUKE UNIVERSITY, Durham (NC); UNIVERSITY OF NORTH CAROLINA AT CHAPEL HILL

(21) Appl. No.: 13/877,281

(22) PCT Filed: Oct. 3, 2011
(86) PCT No.: PCT/US11/01699

§ 371 Date: Apr. 1, 2013

Related U.S. Application Data
(60) Provisional application No. 61/388,711, filed on Oct. 1, 2010.

(51) Int. Cl. G01N 33/68

(52) U.S. Cl. CPC G01N 33/6854
USPC 435/5

(57) ABSTRACT

The present invention relates, in general, to HIV-1 and, in particular, to methods of detecting incident HIV-1 infection.

This application claims priority from U.S. Provisional Application No. 61/388,711, filed Oct. 1, 2010, the entire content of which is incorporated herein by reference.

This invention was made with government support under Grant No. AI067854 and Grant No. AI46725 awarded by the National Institutes of Health. The government has certain rights in the invention.

### TECHNICAL FIELD

The present invention relates, in general, to HIV-1 and, in particular, to methods of detecting incident HIV-1 infection in a subject.

### BACKGROUND

Recent studies of the earliest events following HIV-1 transmission by the transmitted/founder virus demonstrate early destruction of B cell generative microenvironments (Levesque et al, PLoS Med 6:e1000107 (2009)) and that the initial antibody response ineffectively controls virus replication (Tomaras et al, J Virol 82:12449-12463 (2008)). Detailed understanding of the antibody specificities and subclasses elicited after HIV-1 transmission can inform vaccine designs that aim to elicit functional antibodies more readily and more robustly than natural HIV-1 infection. Furthermore, these specific antibody responses may be used as a surrogate of the time since HIV-1 transmission. The latter idea is important because antibody responses, potentially combined with other markers, can be used to define incident HIV infection (Cohen and Fidler, Curr Opin HIV AIDS 5:265-268 (2010)).

Current incidence tests are based on the evolution of the HIV-specific antibody response during the first several months after transmission, where assays measuring the quantity or avidity of HIV-specific antibodies can discriminate recent from chronic infection. One commonly used strategy, the BED EIA, measures the proportion of HIV-1 gp41-specific binding antibodies to total IgG from subtypes B, E, and D by a capture ELISA (Parekh et al, AIDS Res Hum Retroviruses 18:295-307 (2002)). Other strategies, such as the Abbott AxSYM HIV 1/2/gO, use a third generation EIA that exploits the avidity maturation of HIV-specific antibody response to determine time from transmission (Suligoi et al, J Clin Microbiol 40:4015-4020 (2002)). While these assays have been extensively used to determine incidence, they tend to result in a large number of misclassified recent infections and may, therefore, overestimate incidence, especially in non-Clade B populations (Sakarovitch et al, J Acquir Immune Defic Syndr 45:115-122 (2007), Pandori et al, J Clin Microbiol 47:2639-2642 (2009), Karita et al, AIDS 21:403-408 (2007)). In addition, the ability of these assays to accurately distinguish recent infection in individuals on antiretroviral therapy (ART) and with low CD4 counts is difficult (Marinda et al, J Acquir Immune Defic Syndr 53:496-499 (2010), Hladik et al, AIDS Res Hum Retroviruses 27:1-5 (2011)). Additional measurements are needed that could be used in a multi-variate approach to increase specificity in the setting of non-clade B infections and ART use.

Anti-Env HIV-1 plasma antibodies are predominantly IgG1 subclass, whereas anti-Env IgG3 is the second most predominant IgG subclass (Broliden et al, Clin Exp Immunol 76:216-221 (1989)). Antibody effector functions (e.g., complement fixation, Fc receptor binding) are determined by antigen specificity and antibody isotype and subclass and anti-Env IgG1 and IgG3 are predominantly responsible for antibody effector functions (reviewed in Tomaras and Haynes, Curr Opin HIV AIDS 4:373-379 (2009)). Differential regulation of anti-Env and anti-Gag antibodies has been previously described (Binley et al, J Virol 71:2799-2809 (1997)) and anti-Gag IgG3 antibodies were found more frequently in early infection (Klasse and Blomberg, J Infect Dis 156:1026-1030 (1987), McDougal et al, J Clin Invest 80:316-324 (1987), Khalife et al, AIDS Res Hum Retroviruses 4:3-9 (1988), Ljunggren et al, Clin Exp Immunol 73:343-347 (1988)). Moreover, using Western blot analyses, Gag-specific IgG3 was shown to decline 1 to 4 months post-infection (Wilson et al, AIDS 18:2253-2259 (2004)), concordant with a decrease in anti-HIV IgG3 during disease progression (McDougal et al, J Clin Invest 80:316-324 (1987), Ljunggren et al, Clin Exp Immunol 73:343-347 (1988)). Although anti-Gag antibodies do not have known direct antiviral activity, they may be indicative of an active T helper cell response (Binley et al, J Virol 71:2799-2809 (1997)). Furthermore, shorter duration of antigen specific IgG subclasses may reflect inherent differences in antibody subclass durability (i.e. IgG3) or that certain specificities are from predominantly short-lived memory B cells.

The present invention results, at least in part, from studies designed to determine the kinetics of HIV-specific IgG subclass antibody responses in an HIV-1 acute cohort from United States, South Africa, and Malawi. Generally, there was an overall decline in HIV-specific IgG3 responses in all subjects, while HIV-specific IgG1 responses tended to rise and remained elevated throughout the study period. An assessment was made of the applicability of HIV-specific IgG3 antibody responses to determining incidence by applying an exponential decay model to determine the peak IgG3 antibody concentrations as well as the half-life of these antibodies during acute HIV-1 infection (AHI). In addition, the effect of ART use, viral load, and subject location on the peak and half-life of each HIV-specific IgG3 response was also determined. These measurements can form part of a multi-variate algorithm that allows for an estimate of the relative timing from HIV-1 transmission.

### SUMMARY OF THE INVENTION

In general, the present invention relates to HIV-1. More specifically, the invention relates to a method of estimating the relative timing from HIV-1 transmission in a subject.

Objects and advantages of the present invention will be clear from the description that follows.

### BRIEF DESCRIPTION OF THE DRAWINGS

FIGS. 1A and 1B. Levels of HIV-1 specific IgG3 (FIG. 1A) and IgG1 (FIG. 1B) modeled over time during AHI. Antibody responses were modeled over time and response half-life was estimated using the exponential decay model. Half-life in days (95% CI) and N (number of subjects with at least one positive timepoint after peak/number of subjects with a positive response) are depicted in each panel.

FIG. 2. HIV-specific IgG3 antibody responses peak sequentially during AHI. Mean concentrations of HIV-specific IgG3 and IgG1 are shown in a longitudinal model where responses were aligned to study enrollment and adjusted for time post HIV-1 aquisition according to the classification by Fiebig et al, AIDS 17:1871-1879 (2003)).

FIGS. 3A and 3B. Levels of HIV-1 specific IgG1 remain stable while HIV-1 specific IgG3 declines over time during AHI. Declining HIV-1 specific IgG3 antibody responses were aligned according to the peak of each response, modeled over time, and response half-life was estimated using the exponential decay model. Half-life in days (95% CI) and N (number of subjects with at least one positive time point after peak) are depicted in each panel (FIG. 3A). HIV-1 specific IgG1 antibody levels did not decline and, therefore, were not fit to the exponential decay model. For this reason, IgG1 antibody responses were aligned to enrollment into the study (FIG. 3B).

FIG. 4. HIV specific IgG3 responses plotted from enrollment.

### DETAILED DESCRIPTION OF THE INVENTION

In accordance with the present invention, the specific antibody concentrations of all three antigen specific IgG3 responses (gp41, p55Gag and p66 Reverse Transcriptase (RT)) can be used to estimate recent (incident) infection. While tests have been developed for the purpose of incidence testing (Parekh et al, AIDS Res. Hum. Retroviruses 18:295-307 (2002), Janssen et al, JAMA 280:42-48 (1998)), current testing paradigms may not accurately account for variables such as ART, CD4 count, and chronic patient populations (Hallett et al, PLoS One 4:e5720 (2009), Marinda et al, J. Acquir. Immune Defic. Syndr. 53:496-499 (2010), Sakarovitch et al, J. Acquir. Immune Defic. Syndr. 45:115-122 (2007), Barnighausen et al, Epidemiology 21:685-697 (2010), McDougal et al, AIDS Res. Hum. Retroviruses 22:945-952 (2006), Wang and Lagakos, Biometrics (2009)). The present invention, which involves evaluation of p55, gp41 and p66 IgG3 antibody testing, provides an improved method of cross-sectional incidence testing.

Using a cohort of acutely HIV-1 infected subjects in the United States and Malawi, the half-life and concentration of antigen specific IgG3 and IgG1 responses were determined. Also determined was the influence of antiviral therapy and viral load. Estimates for the peak antibody response from the time of infection as well as the antibody half life were obtained using an exponential decay model to determine the concentrations of different HIV-1 specific IgG3 antibodies that allow for an estimate of the relative timing from HIV-1 transmission.

As described in the Examples that follow, a decline in p55Gag-specific, gp4lEnv-specific and p66RT-specific IgG3 responses is observed during AHI with a concurrent maintenance of antigen-specific IgG1. In Example 1, the half-life of the p55Gag IgG3 response was 40.9 days compared with 22.6 days for gp41-specific IgG3. In Example 2, the half-life of the p55Gag IgG3 response was 59.9 days compared with 29 days for gp41-specific IgG3. Using antibody concentrations of Gag, Env and RT at peak, at half-life point and at 150 days, the relative time since transmission can be estimated for an unknown sample.

Thus, the present invention provides a method of determining whether a subject (e.g., a human subject) is recently infected with HIV-1 or is chronically infected. The method comprises obtaining a biological sample (e.g., a plasma sample, serum sample or mucosal fluid) from the subject and determining the HIV-1 specific antibody IgG3 subclass concentrations in the sample for gp41 Env, p66 RT and p55Gag. The concentrations can be determined, for example, using an antibody binding assay, for example, as described in Tomaras et al, J. Virol. 82:12449-12463 (2008) or Yates et al, AIDS 2011 Aug. 9 E-pub ahead of print. For example, the biological sample (e.g., serum, plasma etc) is incubated with the HIV-1 antigens (p66 RT, Gag, gp41 Env) under conditions such that binding can occur. Excess biological material is washed away and IgG3 specific antibody bound to the HIV-1 antigens is detected using a reagent specific for IgG3. The concentration of the IgG3 antibody is determined based on an IgG3 antibody standard. The concentrations of the HIV-1 specific IgG3 responses can be compared to the ranges determined from recent and chronic infection to determine if the biological specimen/sample obtained from the subject has IgG3 concentrations that fall within the window of recent or chronic infection. The IgG3 concentration measurement can be used in combination with other known parameters that either determine HIV infection status (e.g., viral RNA, p24 antigen, HIV-1 specific Ig or IgG1 levels) or are known to distinguish recent from chronic infection (e.g., HIV-1 specific IG avidity). In addition, it has been shown that gp41 IgA declines during acute infection and the half life concentration of IgA antibodies has been determined (see Example 3). Thus, the method described herein can also be practiced using the concentration of gp41 specific IgA antibodies in a biological sample (e.g., serum, plasma, mucosal fluid)

ART use has a significant impact on the half-life of p55 IgG3. As described in the Examples that follow, the antibody response of subjects on ART had a 2-fold longer half-life compared to untreated subjects. Corresponding viral load levels (high viral load/off ART or low viral load/on ART) as well as virus clade (B or C), had similar implications for p55-specific IgG3 half-life and, therefore, their impact cannot be distinguished from that of ART. However, the half-life of p66RT IgG3 was decreased in those on ART, suggesting that virus replication may be a more important factor than CD4⁺ T cell responses in maintaining particular HIV specific antibody responses. In addition, the sequential appearance of gp41-specfic IgG3 followed by p55- and gp140-specific IgG3 is consistent with previous findings involving overall HIV-specific IgG responses (Tomaras et al, J. Virol. 82:12449-12463 (2008)).

In a recent clonal analyses of the initial HIV-1 reactive antibodies, increased levels of IgA and IgG3 antigen-specific antibodies were identified in acute HIV-1 infection when compared to vaccine induced influenza antibodies (Liao et al, Retrovirology 6 (suppl. 3):P73 (2009)). Thus, some of the initial anti-HIV-1 antibody response may be due to stimulation of specific subsets of B cells that preferentially switch from IgM to IgG3, e.g. marginal zone B cells (Gatto et al, J. Immunol. 173:4308-4316 (2004)).

ART resulted in a decrease in the level of HIV-1 gp120 env-specific binding antibody titers compared to untreated subjects (Morris et al, J. Exp. Med. 188:233-245 (1998), Lafeuillade et al, J. Infect. Dis. 175:1051-1055 (1997), Markowitz et al, J. Infect. Dis. 179:527-537 (1999)) and the half-lives were ˜33-81 wk in plasma in antiretroviral drug-treated HIV-1⁺ subjects (Bonsignori et al, J. Immunol. 183:2708-2717 (2009)). However, total IgG antibody responses, mostly comprised of IgG1, to HIV-1 Gag were more durable (Bonsignori et al, J. Immunol. 183:2708-2717 (2009)). Thus, HIV-1 can induce short-lived memory B cell-dependent plasma Abs of particular specificity and IgG subclass after HIV-1 acquisition.

Certain aspects of the invention are described in greater detail in the non-limiting Examples that follows. (See also Yates et al, AIDS. 2011 Aug. 9 E-pub ahead of print.)

### EXAMPLE 1

### Experimental Details

### Subjects

Plasma samples were collected over time from 20 subjects enrolled during acute HIV-1 infection (AHI), each with between 5 and 20 visits (average of 7.9 visits per subject). All subjects were in Fiebig stages 3 through 6 upon enrollment into the CHAVI 001 prospective study (Fiebig et al, J. Acquir. Immune Defic. Syndr. 39:133-137 (2005), McMichael et al, Nat. Rev. Immunol. 10:11-23 (2010)). Twelve of the 20 subjects (60%) were from the United States and, of those, 7 (58.3%) began anti-retroviral therapy (ART) within 4 weeks of enrollment. None of the subjects from Malawi were on ART during the time of this study.

### HIV-1 Multiplex Binding Antibody Assays

Customized multiplex HIV-1 binding assays were performed as previously described to determine IgG1 and IgG3 responses specific for recombinant HIV-1 p55 Gag (Protein Sciences, Meriden, Conn.), recombinant HIV-1 gp41 MN (Immunodiagnostics, Woburn, Mass.), consensus gp140 and gp120 proteins (Dr. Liao, Duke University), HIV-1 p66 RT (Protein Sciences, Meriden, Conn.), HIV-1 recombinant Nef (Genway, San Diego, Calif.), recombinant HIV-1 Tat (Advanced Bioscience, Kensington, Md.), and recombinant HIV-1 p31 Integrase (Genway, San Diego, Calif.) (Tomaras et al, J. Virol. 82:12449-12463 (2008)). HIV-specific antibody subclasses were detected with mouse anti-human IgG1 (BD Pharmingen) and mouse anti-human IgG3 (Calbiochem) on a Bio-Plex instrument (Bio-Rad, Hercules, Calif.) and μg/ml equivalents were calculated using a 4PL curve analysis with IgG subclass standards.

### Statistical Analysis

To obtain estimates of peak antigen values and half-life, exponential decay models were created for each antigen,

y=β₀e^{-x}^{ₜ}^{β}^{₁}

where y is the antigen value, xₜ is the number of days post peak response for the antigen of interest, β₀ is the estimated peak value (level at day 0) and β₁ is the exponential rate of decay such that ln(2)/β₁ is the estimated half-life. The models only include values at or post-peak, assume an asymptote of zero and a random effect for β₀, and were fit using PROC NLMIXED in SAS® 9.2. Approximate 95% confidence intervals for the half-life estimates were generated based on a Student t distribution using the delta method to estimate the variance of the half-life estimates. Specifically,

Var(ln(2)/β₁)=[ln(2)]²Var(β₁)/μ⁴

whereμ equals the estimate of β1 from the model. To explore the impact of ART, viral load and clade status on antigen peak and half-life estimates, additional models were created for each of these status variables that included sub-group specific estimates of peak value and half-life. Specifically,

y=(β_{0,Subgroup1}β_{0.Subgroup2})e^{-x}^{ₜ₍}^{β}^{_{1,Subgroup1}}^{30 β}^{_{1,.Subgroup2}}⁾

where sub-groups are defined as use of ART (yes vs. no), viral load (≦5,000 IU/ml vs. >5,000 IU/ml) and clade (B vs. C) and ART and viral status are time-varying covariates such that values may change for a subject over time. To compare the timing of peak between antigens adjusted mean estimates for each antigen at each visit were obtained from a longitudinal model that adjusts for Fiebig staging at study enrollment in order to account for the differences in the amount of time post-infection upon enrollment. The models only include all observed antigen values, treat each visit as a categorical variable, and were fit using PROCMIXED in SAS® 9.2.

### Results

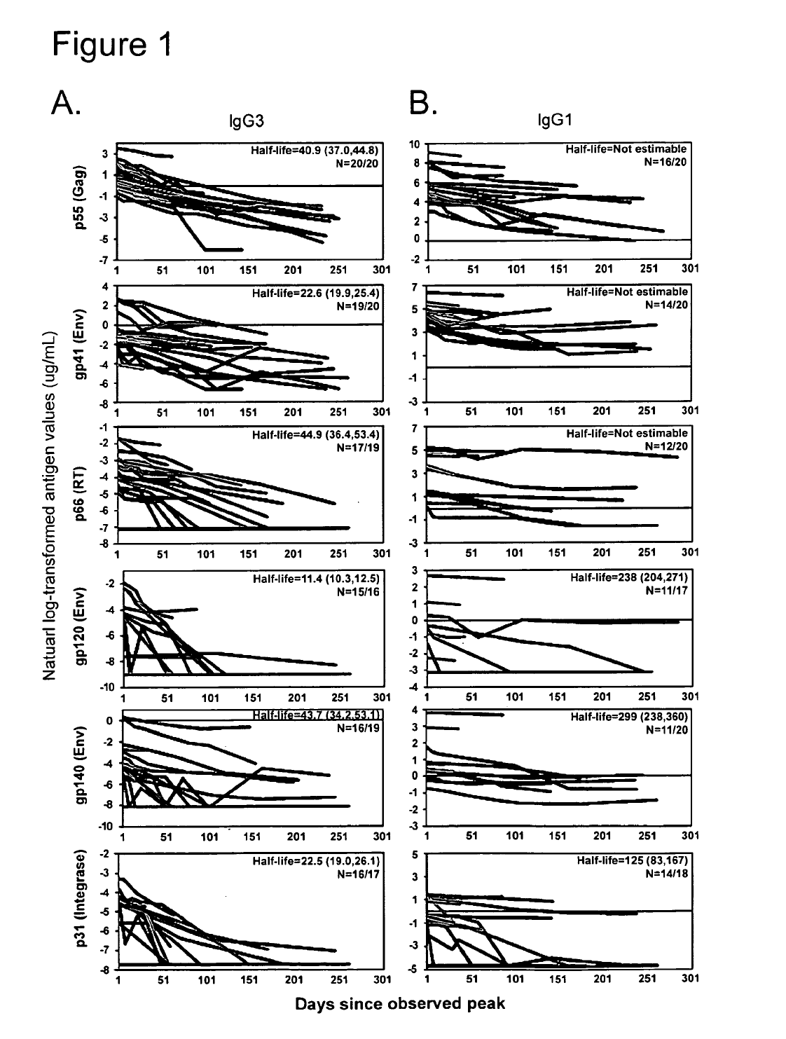

Levels of HIV-specific IgG1 and IgG3 were examined over 45 weeks after enrollment and the half-life (ln(2)/(β₂) of each antibody response was estimated. FIG. 1 shows the kinetics of HIV-specific IgG1 and IgG3 antibodies aligned according to the observed peak of each response. All 20 subjects had detectable p55- and gp41-specific IgG1 and IgG3, and IgG3 steadily declined in all subjects. IgG3 specific for gp140, p66, p31, and gp120 was detected in most (>80%), but not all of the subjects. HIV-1 Nef- and Tat-specific IgG3 were detected in only 50% and 6% of subjects, respectively. Half-life was estimated for all HIV IgG3 antibody responses (except for Nef and Tat, due to insufficient data) and for gp120-, gp140-, and p31-specific IgG1 responses (FIG. 1). In contrast, p55-, gp41-, and p66-specific IgG1 antibodies did not decline.

Estimates for specific antibody concentrations (95% CI) at the observed peak for p55-specific IgG3 and gp41-specific IgG3 were 4.71 (1.09, 8.34) and 2.45 (0.30, 4.60) μg/ml, respectively. The peak titers for the other IgG3 specificities were ≦0.2 μg/ml. The estimated fold decrease (95% CI) from the observed peak response to day 150 post-peak was 12.7 (9.60, 15.80) for p55-specific IgG3 and 99.5 (43.80, 155.20) for gp41-specific IgG3. Estimated antibody concentrations declined to 0.4 μg/ml for p55 IgG3, 0.02 μg/ml for gp41 IgG3 and to 0 μg/ml P66 by 150 days from the peak response.

Antibody responses were also modeled to account for use of antiretroviral therapy (ART), viral load (<=5,000 IU/ml vs. >5,000 IU/ml), and clade (B or C). ART had a profound effect on the half-life estimate of p55 IgG3 responses: subjects off ART had p55 IgG3 responses with a half-life estimate of 21.16 (17.33, 24.99) days and those on ART had a nearly 2-fold longer half-life estimate of 47.73 (43.65,51.81) days. Conversely, ART shortened the half-life estimate of p66 IgG3 responses by over 3-fold: subjects on ART had a half-life estimate of 20.54 (17.97, 23.11) days and those off ART had a half-life estimate of 68.48 (58.64, 78.31) days. ART had no effect on the p31-specific IgG3 half-life estimate. A final model for the effect of ART on the antibody half-life estimate could not be obtained for gp41-, gp120-, gp140-specific IgG3. ART also influenced gp120-specific IgG1 kinetics. Specimens from subjects on ART trended towards a shorter average half-life estimate of 52.7 days (not significant) and those from subjects not on ART showed a slower rate of gp120-IgG1 decline with a half-life estimate of 246.11 (210.46, 281.77) days.

Antibody decay models that included the effect of viral load or virus clade on antibody kinetics showed that viral load and virus clade affected antibody response half-life and peak titer in a fashion similar to that of ART. The only exception was gp120-specific IgG3, where virus clade did have a significant impact although a final model for the impact of ART use could not be obtained. The half-life of gp120-specific IgG3 in clade B subjects was more than 10-fold shorter than in clade C subjects. To assess the post-infection timeframe of the peak of the antibody response, the visit at which the estimated mean peak occurred was obtained from the longitudinal models that adjust for Fiebig staging. It was shown previously that HIV gp41-specific antibodies arise before p55- and gp140-specific antibodies (Tomaras et al, J. Virol. 82:12449-12463 (2008), Tomaras et al, Curr. Opin. HIV AIDS 4:373 (2009)). The adjusted mean titers for HIV-1 gp41-specific IgG3 peaked approximately 1 week before p55 gag IgG3, followed by gp140 and p66 IgG3 2 to 4 weeks later.

### EXAMPLE 2

### Experimental Details

### Subjects

Plasma samples were collected over time from 41 subjects enrolled during acute HIV-1 infection (AHI), each with between 5 and 15 visits. As shown in Table 1, twenty-six of the 41 subjects (63.4%) were from the United States and, of those, 11 (42.3%) began anti-retroviral therapy (ART) within 4 weeks of enrollment. An additional two subjects from the USA began ART at 12 and 16 weeks after enrollment. No subjects from South Africa/Malawi were on ART while plasma samples were being collected. Fifteen (36.5%) of subjects were white and 5 (12.2%) were female. Subjects' ages ranged from 17 to 60 years with an average age of 28.4 years. HIV-1 clade typing was done on a subset of subjects at enrollment and virus clade corresponded to location (Clade B=North America, Clade C=Africa) for each subject. CD4 counts were done on most subjects, but data was not obtained for all study visits (Table 2). Viral load levels were also measured for most visits for each subject (Table 3) and viral load set point was determined by averaging all viral load measurements within a defined set point window (Fellay et al, Science 317:944-947 (2007)). All subjects were assigned to Fiebig stages 1 through 6 upon enrollment into the CHAVI 001 prospective study (Table 4) (Fiebig et al, J Acquir Immune Defic Syndr 39:133-137 (2005), McMichael et al, Nat Rev Immunol 10:11-23 (2010), Fiebig et al, AIDS 17:1871-1879 (2003)). Upon enrollment, 4 subjects were in Fiebig stages I-II (viral RNA positive only in stage 1 and viral RNA and p24 antigen positive in stage II, antibody EIA non-reactive and occurring approximately 10-24.3 days from transmission); 4 subjects were in Fiebig stage III (viral RNA, p24 antigen, EIA positive, Western blot negative and occurring approximately 20.3-27.5 days from transmission); 11 subjects were in Fiebig stage IV (viral RNA, p24 antigen, EIA positive, Western blot indeterminate and approximately 23.5-33.1 days from transmission), and the rest of the subjects (Fiebig et al, AIDS 17:1871-1879 (2003)) were in Fiebig stages V to VI (viral RNA, p24 antigen, ELISA positive, Western blot positive, p31 negative to positive in stage VI, with stage V occurring approximately 29.1 to 102.6 days from transmission).

TABLE 1
Clinical data for CHAVI 001 subjects
CHAVI 001 Subjects n = 41

| | |
|---|---|
| Black/White/Unknown | 25/15/1 |
| Mean Age (range) | 28.4 (17-60) |
| Male/Female | 36/5 |
| USA/Malawi/South Africa | 26/11/4 |
| On ART/Off ART* | 13/28 |

*13 subjects began ART at some point prior to the last sample collection, with 11 subjects starting ART within 4 weeks of enrollment. Some subjects had interrupted ART during the study.

TABLE 2
CD4 counts for CHAVI 001 subjects

| | Study Week | | | | | | | | | | | | | | |
|---|---|---|---|---|---|---|---|---|---|---|---|---|---|---|---|
| ptid | Scr | Enr | 1 | 2 | 3 | 4 | 8 | 12 | 16 | 24 | 36 | 48 | 60 | 72 | 84 |
| n (out of 41) | 2 | 39 | 0 | 8 | 5 | 8 | 1 | 31 | 11 | 39 | 35 | 36 | 30 | 28 | 27 |
| average | 607 | 504 | N/A* | 563 | 512 | 526 | 688 | 649 | 642 | 570 | 585 | 607 | 608 | 624 | 644 |
| std dev | 85 | 253 | N/A | 178 | 214 | 126 | 0 | 256 | 117 | 219 | 259 | 344 | 272 | 215 | 257 |

*N/A—Not Available

| | | | Study Week | | |
|---|---|---|---|---|---|
| Subject | Screening | Enrollment | Week 1 | Week 2 | Set point |
| C1-00-001-9 | 52695 | 741499 | 418672 | 311181 | 371535 |
| C1-00-005-8 | 92581 | 394649 | | 181169 | 234 |
| C1-00-008-1 | 828693 | 3746 | 104 | <400 | N/A* |
| C1-00-010-6 | >750000 | | 14538865 | 265006 | N/A |
| C1-00-015-0 | 127809 | 589437 | 36405 | 30424 | N/A |
| C1-00-023-8 | >100000 | 596908 | 3546 | 985 | N/A |
| C1-00-025-2 | | 12803 | 12140 | 5225 | N/A |
| C1-00-029-5 | 595942 | 92534 | 43807 | 285388 | N/A |
| C1-00-034-1 | 23456427 | 5076826 | 677972 | 21921 | N/A |
| C1-00-043-9 | 70500 | 31402 | 7813 | <400 | N/A |
| C1-00-047-0 | 84193 | 264882 | | | 23442 |
| C1-00-058-4 | 1730000 | 523087 | 74814 | 7417 | N/A |
| C1-00-060-7 | 8009 | 2837 | 8972 | 16893 | N/A |
| C1-00-061-0 | <400 | 123928 | 3893 | 377 | N/A |
| C1-00-065-4 | 6899055 | 2346147 | 189930 | 95419 | N/A |
| C1-00-070-0 | 936 | 1521 | 1288 | 7268 | 1413 |
| C1-01-005-5 | >750000 | 31513812 | 22826172 | 126687 | N/A |
| C1-01-019-9 | 126500 | 31196 | 32517 | 15494 | N/D* |
| C1-01-022-2 | | 53507 | 34883 | 26353 | 4365 |
| C1-01-024-8 | 1899 | 2485 | 3144 | 2283 | N/D |
| C1-03-001-0 | 12995 | 408727 | 287756 | 489476 | 181970 |
| C1-03-005-4 | 14225 | 13936 | 9241 | 13158 | 12303 |
| C1-03-013-1 | 411873 | 437369 | 9366 | 7764 | 22909 |
| C1-03-025-6 | 62060 | 254060 | | 17990 | 18621 |
| C1-03-027-5 | 410499 | 415450 | 1420575 | 477910 | 87096 |
| C1-03-042-7 | 620294 | 1644231 | 239554 | 139119 | 93325 |
| C1-03-045-5 | 963808 | 502665 | 354067 | 443894 | N/D |
| C1-03-068-1 | 711695 | 106742 | 9734 | | 25119 |
| C1-03-069-4 | 2809692 | 243636 | 36531 | 20192 | 77625 |
| C1-03-082-2 | 621441 | 452850 | 140381 | 124245 | 194984 |
| C1-03-085-0 | 83378 | 1200417 | 900179 | 510384 | 15488 |
| C1-04-005-6 | 2270 | 3830 | 3730 | | 6761 |
| C1-05-014-9 | >10000000 | 5640174 | 1972828 | 1941017 | 93325 |
| C1-05-035-8 | >1000000 | 3333584 | 9962123 | 3664505 | 575440 |
| C1-05-051-7 | >1000000 | 663919 | 1011724 | 1476197 | 181970 |
| C1-16-002-1 | 2387 | 22300 | | | N/D |
| C1-16-003-6 | 28137 | 6110 | 4710 | 5280 | 8913 |
| C1-16-005-4 | 214000 | 65300 | 87400 | 41700 | 61660 |
| C1-16-013-1 | 52967 | 105332 | 56099 | | 13183 |
| C1-16-019-3 | 2830 | 2375 | 542 | | 38019 |
| C1-16-030-8 | | 1974 | 1687 | 2019 | 1778 |

*N/A—Not Available because subject began ART within 6 months after enrollment
^{#}N/D—Not Determined

TABLE 4
Fiebig staging data for CHAVI 001 subjects

| Fiebig | Approximate timeframe post | |
|---|---|---|
| Stage | transmission (days)* | # Subjects |
| I-II | 10-24.3 | 4 |
| III | 20.3-27.5 | 4 |
| IV | 23.5-33.1 | 11 |
| V-VI | Stage V = 29.1-102.6 | 22 |
| | Stage VI = 98.6-open-ended | |

*Derived from Fiebig et al 2003, allowing 10-14 days for the eclipse phase

This CHAVI Acute and Chronic Cohorts study was reviewed and approved by the institutional review boards of Duke University Medical Center. All participants provided written informed consent for study participation.

### HIV-I Multiplex Binding Antibody Assays

Customized multiplex HIV-1 binding assays were performed as previously described (Tomaras et al, J Virol 82:12449-12463 (2008)) to determine IgG1 and IgG3 responses specific for recombinant HIV-1 p55 Gag (Protein Sciences, Meriden, Conn.), recombinant HIV-1 gp41 MN (Immunodiagnostics, Woburn, Mass.), a previously-described artificial multi-clade group M consensus gp120 Env protein (Con6 gp120) (Drs. Liao and Haynes, Duke University) (Gao et al, J Virol 79:1154-1163 (2005)), HIV-1 p66 reverse transcriptase (RT) (Protein Sciences, Meriden, Conn.), HIV-1 recombinant Nef (Genway, San Diego, Calif.), recombinant HIV-1 Tat (Advanced Bioscience, Kensington, Md.), and recombinant HIV-1 p31 Integrase (Genway, San Diego, Calif.) (Tomaras et al, J Virol 82:12449-12463 (2008)). HIV-specific antibody subclasses were detected with mouse anti-human IgG1 (BD Pharmingen) and mouse anti-human IgG3 (Calbiochem) on a Bio-Plex instrument (Bio-Rad, Hercules, Calif.) and μg/ml equivalents were calculated using a 4PL curve analysis with IgG subclass standards. Mouse anti-human IgG1 and IgG3 detection antibodies were tested for cross-reactivity to IgG1, IgG2, IgG3, and IgG4 and found to be highly specific for subclass detection. HIVIG (Quality Biological) and a constant HIV+ serum titration were utilized as positive controls and negative controls were included in every assay. All assays were run under GCLP compliant conditions, including tracking of positive controls by Levy-Jennings charts. Positivity criteria (mean MFI+3 STDEV) for antibody-antigen pairs were predetermined using a set of plasmas from 30 seronegative subjects. FDA compliant software, Bio-Plex Manager 5.0, (BioRad, Hercules, Calif.) was utilized for the analysis of specimens.

### Statistical Analysis

To obtain estimates of peak antigen values and half-life, exponential decay models were created for each antigen,

y=β₀e^{-x}^{ₜ}^{β}^{₁}

where y is the antigen value, xₜ is the number of days post observed peak response for the antigen of interest, β₀ is the estimated peak value (level at day 0) and β₁ is the exponential rate of decay such that ln(2)/β₁ is the estimated half-life. The models only include values at or post-peak, assume an asymptote of zero and a random effect for β₀, and were fit using PROC NLMLXED in SAS® 9.2. Approximate 95% confidence intervals for the half-life estimates were generated based on a Student t distribution using the delta method to estimate the variance of the half-life estimates. Specifically,

Var(ln(2)/β₁)=[ln(2)]²Var(β₁)/μ⁴

where μ equals the estimate of β1 from the model. To explore the impact of ART, viral load and subject location status on antigen peak and half-life estimates, additional models were created for each of these status variables that included sub-group specific estimates of peak value and half-life. Specifically,

y=(β_{0,Subgroup 1}+β_{0,Subgroup 2})e^{-x}^{ₜ}^{(β}^{_{1,Subgroup 1}}^{+β}^{_{1,Subgroup 2}}⁾

where sub-groups are defined as use of ART (yes vs. no), viral load (≦5,000 IU/ml vs. >5,000 IU/ml) and subject location (USA vs. South Africa/Malawi) and ART and viral status are time-varying covariates such that values may change for a subject over time. Because ART was only used in subjects from the USA, the ART models included only the USA subjects. Likewise, the subject location models included only observations prior to ART initiation. To compare the timing of peak between antigens adjusted mean estimates for each antigen at each visit were obtained from a longitudinal model that adjusts for Fiebig staging at study enrollment in order to account for the differences in the amount of time post-infection upon enrollment. The models include all observed antigen values, treat each visit as a categorical variable, and were fit using PROCMIXED in SAS® 9.2.

### Results

To evaluate the magnitude and kinetics of IgG subclass responses to acute HIV infection, HIV-specific IgG1 and IgG3 antibody responses were measured in 41 individuals in the CHAVI 001 cohort. Table 5 shows the seroprevalence of HIV-specific IgG1 and IgG3 antibodies in these individuals. HIV-1 p55 Gag- and gp41 Env-specific IgG1 was detected in all subjects, and IgG1 specific for p66 RT, p31 Integrase, and gp120 Env was detected in at least 85% of subjects. All 41 subjects had detectable p55- and gp41-specific IgG3, and IgG3 specific for p66 RT, p31 Integrase, and gp120 Env was detected in most (>80%), but not all of the subjects. HIV-1 Nef- and Tat-specific IgG3 were detected in only 16 subjects (39%) and 20 subjects (49%), respectively. These data show that IgG1 and IgG3 antibodies directed toward p55 Gag, gp41 Env, gp120 Env, p66 RT, and gp120 Env are detectable in most subjects during AHI.

TABLE 5
Seroprevalence of HIV-specific IgG1 and IgG3 Antibodies During AHI

| | IgG1 n = 41 | IgG3 n = 41 |
|---|---|---|
| | Positive # (%) | Positive # (%) |
| p55 Gag | 41 (100) | 41 (100.0) |
| gp41 Env | 41 (100) | 41 (100.0) |
| p66 RT | 40 (97.6) | 39 (95.1) |
| p31 Integrase | 37 (90.2) | 33 (80.5) |
| gp120 Env | 35 (85.4) | 35 (85.4) |
| Tat | 9 (22.0) | 20 (48.8) |
| Nef | 19* (50.0) | 16 (39.0) |

*n = 38

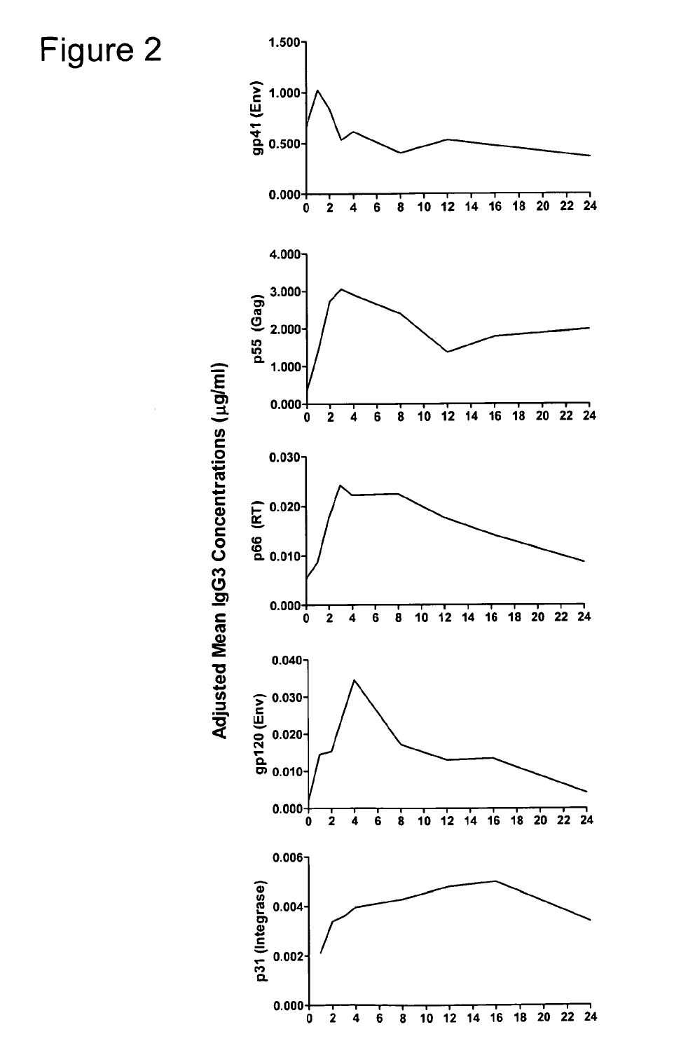

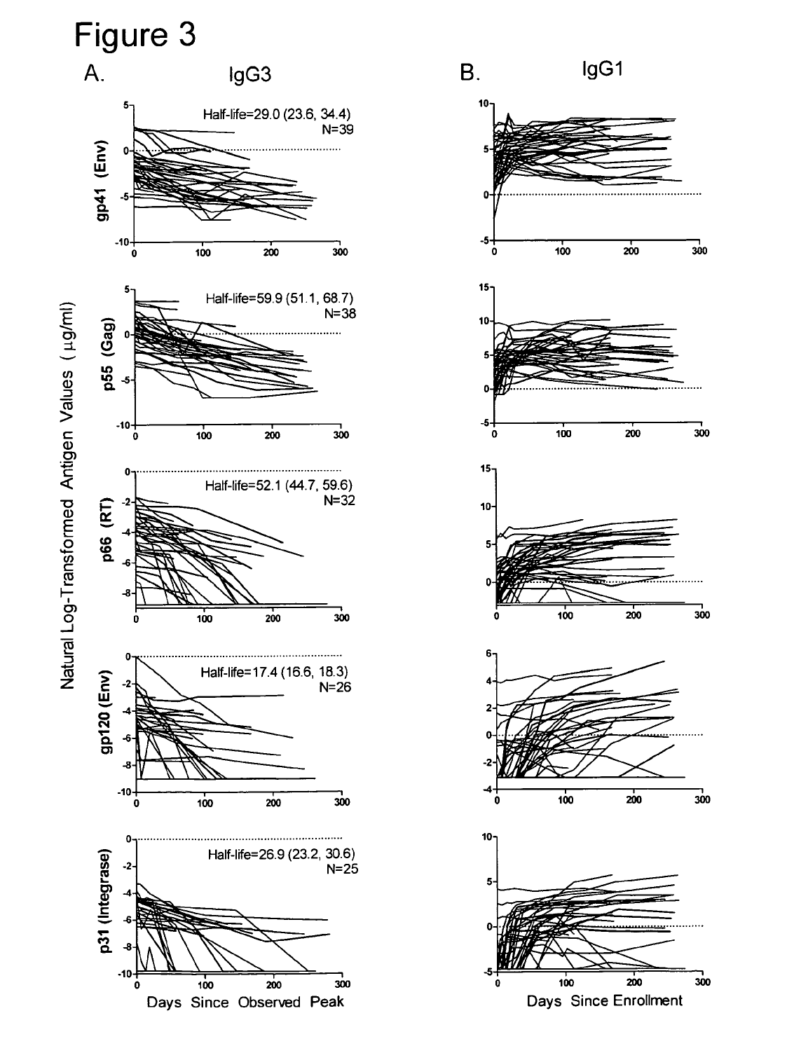

It was previously shown that HIV 41-specific antibodies are the first to arise during AHI and they are followed by p55 Gag-, p66 RT-, gp120 Env- and p31 Integrase-specific antibodies (Tomaras et al, J Virol 82:12449-12463 (2008), Tomaras and Haynes, Curr Opin HIV AIDS 4:373-379 (2009)). To assess the post-infection timeframe of the peak of the IgG3 and IgG1 antibody response, the visit at which the estimated mean peak occurred was obtained from the longitudinal models that adjust for Fiebig staging (since subjects were enrolled at different Fiebig stages). The adjusted mean titers for HIV-1 gp41 Env-specific IgG3 peaked approximately 1 week post-enrollment, followed by p55 Gag- and p66 RT-specific IgG3 at 3 weeks post-enrollment, gp120 Env-specific IgG3 at 4 weeks post-enrollment, and p31 Integrase-specific IgG3 at 16 weeks post-enrollment (FIG. 2). HIV-specific IgG1 antibodies did not show an overall discernable peak, (FIG. 3B) These results show that, in contrast to HIV-specific IgG1, HIV-specific IgG3 antibodies decline over time and the timing of the peak of individual HIV-specific IgG3 antibody responses is a reflection of their sequential elicitation during AHI.

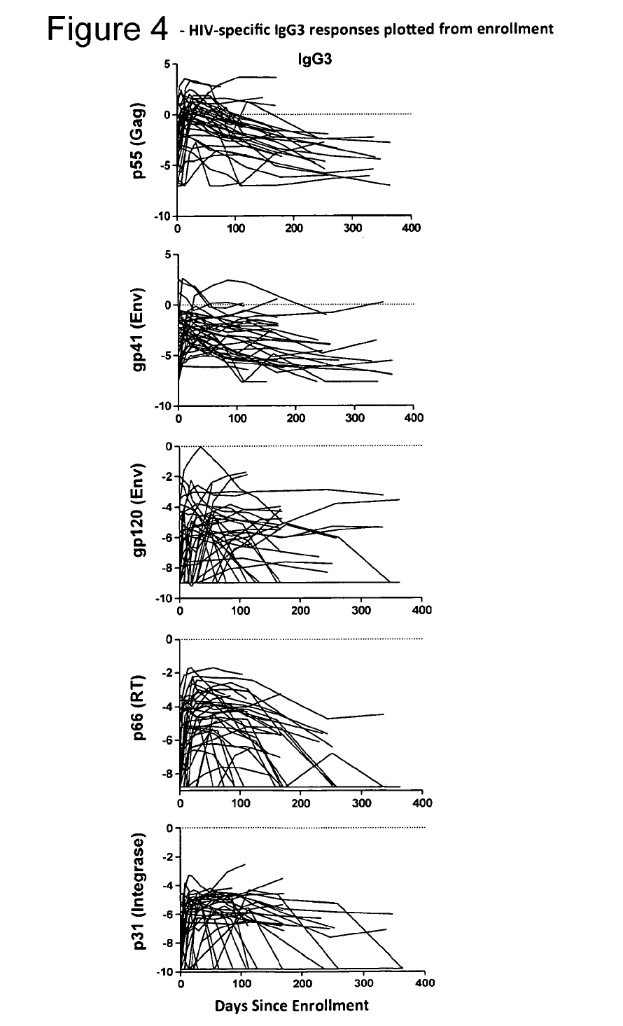

Levels of HIV-specific IgG1 and IgG3 antibodies were also examined through 43 weeks after enrollment and models were attempted to estimate the half-life (ln(2)/β₂) of each antibody response. FIG. 3 shows the kinetics of HIV-specific IgG1 and IgG3 antibody responses during AHI. HIV-specific IgG3 showed an overall decline, while HIV-specific IgG1 antibody levels remained more stable and thus would not fit the exponential decay model. Therefore, half-life of the HIV-specific IgG1 antibody responses for all responding subjects could not be estimated and as such, these responses are aligned by enrollment instead of peak response as shown for HIV-specific IgG3 (FIG. 3). In FIG. 4, HIV-specific IgG3 antibody responses are aligned to enrollment and, therefore, the rise and decline of these responses can be observed for individual subjects. Using the exponential decay model, half-life was estimated for p55 Gag-, gp41 Env-, gp120 Env-, p31 Integrase- and p66 RT-specific IgG3 antibody responses, with gp120 Env-specific IgG3 having the shortest half-life estimate of 17.4 (95% CI=16.6, 18.3) days and p55 Gag-specific IgG3 having the longest half-life estimate of 59.9 (95% CI=51.1, 68.7) days (FIG. 3 and Table 6). Because Nef and Tat IgG3 antibody responses were detected in less than half of all subjects and would, therefore, not be very useful to determine incidence, the results of the exponential decay model are not shown. These results demonstrate that HIV-specific IgG3 antibodies tend to decline during AHI in a defined mariner while HIV-specific IgG1 antibodies tend to remain stable or elevate over time.

TABLE 6
Effects of ART use, viral load, and virus clade on IgG3 antibody half-life

| Antibody | | | Half-Life (days) | | |
|---|---|---|---|---|---|
| Response | Model | Estimate Type | Estimate | 95% CI | P-valu [symbol] |
| IgG3 p55 | Exponential Decay Model | Overall | 59.91 | 51.05, 68.74 | |
| | Viral Load Only Model | Low | 49.09 | 36.19, 61.99 | 0.009 [symbol] |
| | | High | 77.24 | 60.85, 93.63 | |
| | ART Use Only Model* | No | 22.10 | 17.57, 26.65 | <0.00 [symbol] |
| | | Yes | 47.40 | 44.37, 50.44 | |
| | Location Only Model\hat{ } | USA | 13.59 | 0.70, 26.49 | 0.092 [symbol] |
| | | Africa^{#} | 71.71 | 56.42, 87.01 | |
| IgG3 p66 | Exponential Decay Model | Overall | 52.14 | 44.67, 59.61 | |
| | Viral Load Only Model | Low | 42.41 | 33.39, 51.43 | 0.011 [symbol] |
| | | High | 62.7 | 49.96, 75.44 | |
| | ART Use Only Model* | No | 50.11 | 44.09, 56.12 | <0.00 [symbol] |
| | | Yes | 20.54 | 18.04, 23.05 | |
| | Location Only Model\hat{ } | USA | 51.07 | 40.20, 61.93 | 0.014 [symbol] |
| | | Africa | 74.66 | 60.23, 89.10 | |
| IgG3 p31 | Exponential Decay Model | Overall | 26.86 | 23.18, 30.55 | |
| | Viral Load Only Model | Low | 25.04 | 19.84, 30.23, | 0.239 [symbol] |
| | | High | 29.47 | 23.67, 35.27 | |
| | ART Use Only Model* | No | 33.12 | 23.33, 42.92 | 0.004 [symbol] |
| | | Yes | 20.44 | 16.84, 24.05 | |
| | Location Only Model\hat{ } | USA | 33.08 | 22.9, 43.26 | 0.003 [symbol] |
| | | Africa | 40.57 | 28.50, 52.64 | |
| IgG3 gp41 | Exponential Decay Model | Overall | 28.99 | 23.56, 34.42 | |
| | Location Only Model\hat{ } | USA | 70.16 | 42.16, 98.15 | <0.00 [symbol] |
| | | Africa | 24.96 | 19.13, 30.78 | |
| IgG3 gp120 | Exponential Decay Model | Overall | 17.43 | 16.58, 18.28 | |
| | Viral Load Only Model | Low | 11.65 | 7.23, 16.06 | 0.014 [symbol] |
| | | High | 17.57 | 16.83, 18.30 | |
| | ART Use Only Model* | No | 17.49 | 17.15, 17.83 | <0.00 [symbol] |
| | | Yes | 11.33 | 10.41, 12.26 | |
| | Location Only Model\hat{ } | USA | 17.50 | 17.05, 17.94 | <0.000 [symbol] |
| | | Africa | 529.95 | 183.6, 876.3 | |

*USA subjects only
^{#}Malawi/South Africa = predominantly clade C
\hat{ }Subjects not on ART only or analysis truncated before start of ART [symbol]
indicates data missing or illegible when filed

The exponential decay model was also used to obtain estimates for specific antibody concentrations (95% CI) at the observed peak for p55 Gag-specific IgG3 and gp41-specific IgG3, which were 4.67 (1.76, 7.57) and 1.49 (0.37, 2.62) μg/ml, respectively (Table 7). The peak titers for the other IgG3 specificities were ≦0.1 μg/ml. The estimated fold decrease (95% CI) from the observed peak response to day 150 post-peak was 5.7 (4.22, 7.12) for p55 Gag-specific IgG3 and 36.11 (11.87, 60.35) for gp41 Env-specific IgG3. Estimated antibody concentrations declined to 0.82 μg/ml for p55 Gag-specific IgG3and 0.04 μg/ml for gp41 Env-specific IgG3 by 150 days from the peak response. Together with the half-life values obtained from the exponential decay model, these data show a measurable decline in HIV-specific IgG3 antibodies during AHI.

TABLE 7
IgG3 Concentration estimates from the exponential decay model

| | Concentration at | | Concentration at Half- | | Concentration at 150 Days | | Fold Decrease at [symbol] | |
|---|---|---|---|---|---|---|---|---|
| | Peak (μg/ml) | | Life (μg/ml) | | Post-Peak (μg/ml) | | Post-Pea [symbol] | |
| Specificity | Est. | 95% Cl | Est. | 95% Cl | Est. | 95% Cl | Est. | 95% C [symbol] |
| p55 Gag | 4.67 | 1.76, 7,57 | 2.33 | 0.88, 3.78 | 0.82 | 0.28, 1.37 | 5.67 | 4.22, 7 [symbol] |
| gp41 Env | 1.49 | 0.37, 2.62 | 0.75 | 0.18, 1.31 | 0.04 | 0.00, 0.08 | 36.11 | 11.87, [symbol] |
| p66 RT | 0.03 | 0.02, 0.05 | 0.0161 | 0.01, 0.02 | 0.0044 | 0.0022, 0.0065 | 7.35 | 5.25, 9. [symbol] |
| gp120 Env | 0.05 | 0.00, 0.10 | 0.03 | 0.0022, 0.05 | 0.0001 | 0.00, 0.0003 | 389.80 | 276.24, [symbol] |
| p31 | 0.01 | 0.01, 0.01 | 0.0048 | 0.0027, 0.0069 | 0.0002 | 0.0001, 0.0003 | 47.97 | 22.5, 73 [symbol] |

[symbol]

indicates data missing or illegible when filed

Antibody responses were also modeled to account for the effects of antiretroviral therapy (ART) use, viral load (<=5,000 IU/ml vs. >5,000 IU/ml), and location (USA, predominantly Clade B or South Africa/Malawi, predominantly Clade C) on the half-life (Table 6) and peak concentration (Table 8) of HIV-specific IgG3 responses. To separate any possible effects of ART from location on antibody half-life, only subjects from the USA were evaluated for the effects of ART on IgG3 responses (because all but one of the subjects on ART were in the USA). ART significantly lengthened the half-life estimate of p55 Gag-specific IgG3 responses: subjects off ART had p55 Gag-specific IgG3 responses with a half-life estimate of 22.10 (17.57, 26.65) days and those on ART had a longer half-life estimate of 47.40 (44.37, 50.04) days. Because of the variation in the kinetics of gp41 Env-specific IgG3 responses, the effects of ART on the half-life of this response could not be ascertained. In contrast to its effects on the half-life of p55 Gag-specific IgG3, ART significantly shortened the half-life estimate of p66 RT-specific IgG3 responses by over 2-fold: subjects on ART had a half-life estimate of 20.54 (18.04, 23.05) days and those off ART had a half-life estimate of 50.11 (44.09, 56.12) days (Table 6). ART also significantly shortened the half-lives of p31 Integrase-specific IgG3 and gp120 Env-specific IgG3 by 1.6- and 1.5-fold, respectively. ART use significantly increased the peak concentration of gp120 Env-specific IgG3 responses, but did not affect the peak concentrations of any of the other IgG3 responses (Table 8). Also, because HIV-specific IgG1 antibodies did not decline during AHI and the exponential decay model could not be applied, the influence of ART on the kinetics of these responses was not determined. In summary, ART use tends to prolong the IgG3 antibody response to p55 Gag, but shortens the IgG3 antibody response to p66 RT, p31 Integrase, and gp120 Env.

TABLE 8
Effects of ART use, viral load, and virus clade on peak Ig

[symbol]

| | | | Peak Concentration | | |
|---|---|---|---|---|---|
| Antibody | | | (μg/ml) | | |
| Response | Model | Estimate Type | Estimate | 95% CI | P-v [symbol] |
| IgG3 p55 | Exponential Decay Model | Overall | 4.67 | 1.76, 7.57 | |
| | Viral Load Only Model | Low | 10.3 | 5.34, 15.26 | 0.2 [symbol] |
| | | High | 11.93 | 7.40, 16.47 | |
| | ART Use Only Model* | No | 3.52 | 0.70, 6.34 | 0. [symbol] |
| | | Yes | 2.52 | −0.33, 5.37 | |
| | Location Only Model\hat{ } | USA | 2.43 | −1.07, 5.93 | 0. [symbol] |
| | | Africa^{#} | 7.71 | 3.50, 11.91 | |
| IgG3 p66 | Exponential Decay Model | Overall | 0.03 | 0.02, 0.05 | |
| | Viral Load Only Model | Low | 0.05 | 0.03, 0.07 | 0. [symbol] |
| | | High | 0.06 | 0.04, 0.08 | |
| | ART Use Only Model* | No | 0.03 | 0.01, 0.05 | 0. [symbol] |
| | | Yes | 0.03 | 0.01, 0.06 | |
| | Location Only Model\hat{ } | USA | 0.02 | 0.01, 0.04 | 0.2 [symbol] |
| | | Africa | 0.04 | 0.02, 0.06 | |
| IgG3 p31 | Exponential Decay Model | Overall | 0.01 | 0.01, 0.01 | |
| | Viral Load Only Model | Low | 0.01 | 0.01, 0.07 | 0. [symbol] |
| | | High | 0.01 | 0.01, 0.01 | |
| | ART Use Only Model* | No | 0.01 | 0.00, 0.02 | 0. [symbol] |
| | | Yes | 0.01 | 0.00, 0.02 | |
| | Location Only Model\hat{ } | USA | 0.01 | 0.00, 0.01 | 0.7 [symbol] |
| | | Africa | 0.01 | 0.00, 0.02 | |
| IgG3 gp41 | Exponential Decay Model | Overall | 1.49 | 0.37, 2.62 | |
| | Location Only Model\hat{ } | USA | 1.10 | −0.46, 2.66 | 0.3 [symbol] |
| | | Africa | 2.32 | 0.41, 4.23 | |
| IgG3 gp120 | Exponential Decay Model | Overall | 0.05 | 0.00, 0.10 | |
| | Viral Load Only Model | Low | 0.01 | 0.00, 0.03 | 0.0 [symbol] |
| | | High | 0.03 | 0.01, 0.04 | |
| | ART Use Only Model* | No | 0.04 | −0.04, 0.12 | 0.0 [symbol] |
| | | Yes | 0.09 | 0.00, 0.17 | |
| | Location Only Model\hat{ } | USA | 0.05 | −0.02, 0.12 | 0.8 [symbol] |
| | | Africa | 0.04 | −0.04, 0.13 | |

*USA subjects only
^{#}Malawi/South Africa = predominantly clade C
\hat{ }Subjects not on ART only or analysis truncated before start of ART [symbol]
indicates data missing or illegible when filed

The effects of viral load and location on IgG3 antibody half-life and peak concentration were also evaluated (Table 6). All 41 subjects were analyzed for the effects of viral load on the IgG3 response. Subjects with a viral load of >5000 IU/ml had a significantly longer IgG3 antibody half-life for responses to p55 Gag, p66 RT, and gp120 Env. Viral load levels did not have an effect on the half-life of p31 Integrase-specific IgG3 and a final model could not be obtained for the effects of viral load on gp41 Env-specific IgG3 half-life. Viral load levels did not significantly affect the peak concentration of any of the HIV-specific IgG3 antibody responses (Table 8).

For the evaluation of the effects of subject location on IgG3 antibody half-life and peak concentration, only observations prior to ART initiation were analyzed. This eliminates any possible influence of ART use that may interfere with the effects of location on IgG3 kinetics. It was observed that subjects in South Africa/Malawi had significantly longer antibody half-lives for p66 RT, p31 Integrase, and gp120 Env IgG3 compared to subjects in the USA (Table 6). Subjects in South Africa/Malawi also had a longer antibody half-life for p55 Gag-specific IgG3, but the difference between antibody half-lives in subjects in the U.S. vs. South Africa/Malawi was not significant. However, subjects in South Africa/Malawi had a shorter gp41 Env-specific antibody half-life compared to subjects in the USA. There were no location-specific effects on the peak of IgG3 antibody responses (Table 8). Together, these results show that subjects with viral load levels above 5000 IU/ml had longer IgG3 antibody half-lives for p55 Gag-, p66 RT- and gp120 Env-specific responses. In addition, subjects in Africa had p66RT-, p31 Integrase-, and gp120 Env-specific IgG3 responses that were of significantly longer duration compared to subjects in the USA.

In summary, the HIV-specific IgG1 and IgG3 responses to AHI have been examined in a defined manner and it is proposed that these measurements can be used in an algorithm to determine incident HIV-1 infection. The focus was maintained on IgG1 and IgG3 subclasses, since HIV-specific IgG2 antibodies are not detected in most individuals and the elicitation of HIV-specific IgG4 may be delayed in comparison (reviewed in Tomaras and Haynes, Curr Opin HIV AIDS 4:373-379 (2009)). It has been found that, while IgG1 and IgG3 responses are elicited to gp41 Env, p55 Gag, p66 RT, gp120 Env, and p31 Integrase in a majority of subjects, IgG3 responses to these antigens typically decline. In contrast, HIV-1 IgG1 antibody levels are typically maintained over the same time period. The half-life of the HIV-specific IgG3 antibody response was estimated and concentrations of these antibodies at their peak and nadir (approximately 150 days post-peak) were determined.

ART use was found to have a significant impact on the half-life of p55 IgG3, where the antibody response of subjects on ART had a 2-fold longer half-life compared to untreated subjects. Though there was not enough CD4 count data available to make an association in this study, this finding is consistent with the idea that the Gag-specific antibody response may be more dependent on CD4 T cell help than responses to other HIV-1 antigens (Binley et al, J Virol 71:2799-2809 (1997)). In contrast to p55 Gag-specific IgG3, the half-life of p66 RT-, p31 integrase-, and gp120 Env-specific IgG3 was decreased in subjects on ART, suggesting that virus replication may be a more important factor than CD4⁺ T cell responses in maintaining particular HIV specific antibody responses. There was not enough CD4 count data available during visits where the peak and subsequent initial decline of IgG3 antibodies was observed to significantly determine the effect of CD4 counts on specific antibody responses. Subjects in Malawi/South Africa had longer IgG3 antibody half-lives than subjects in the USA for p66 RT, p31 integrase, and gp120 Env-specific responses. A similar, though non-significant, trend was also observed for p55 Gag-specific IgG3 half-life., Subject location had the opposite effect on gp41 Env-specific IgG3 half-life. Since only samples prior to ART initiation were evaluated in this particular analysis, these data suggest that there are location-specific effects on the duration of HIV-specific IgG3 responses. Importantly, there did not appear to be any significant clade-specific differences in the ability of the antigens used to detect HIV-specific antibodies in the subjects. All 15 subjects in Malawi/South Africa (predominantly Clade C) were positive for p66 RT-, p31 Integrase-, and gp120 Env-specific IgG3 (data not shown). This is not surprising given that p66 RT and p31 Integrase are relatively conserved proteins and the gp120 Env used was an artificial multi-clade group M consensus gp120 Env protein (Con6 gp120) (Gao et al, J Virol 79:1154-1163 (2005)).

In a recent clonal analyses of the initial HIV-1 reactive antibodies, increased levels of IgA and IgG3 antigen- specific antibodies were identified in acute HIV-1 infection when compared to vaccine-induced influenza antibodies (Liao et al, Retrovirology 6(suppl 3):73 (2009)). Thus, some of the initial anti-HIV-1 antibody response may be due to stimulation of specific subsets of B cells that preferentially switch from IgM to IgG3, e.g. marginal zone B cells (Gatto et al, J Immunol 173:4308-4316 (2004)). In addition, the sequential appearance of gp41-specfic IgG3 followed by p55 Gag- and gp120 Env-specific IgG3 is consistent with previous findings involving overall HIV-specific IgG responses (Tomaras et al, J Virol 82:12449-12463 (2008)). These results are also consistent with the finding that the initial HIV-1 gp41 Env-specific response is due to stimulation of a pre-existing pool of cross-reactive memory B cells that had been previously activated by non-HIV-1 antigens (Liao et al, submitted).

Using the antibody concentrations of Gag, Env and RT at peak, at half-life point and at 150 days, the relative time since transmission could be estimated for an unknown sample. Strategies have been developed for the purpose of incidence testing (Parekh et al, AIDS Res Hum Retroviruses 18:295-307 (2002), Janssen et al, JAMA 280:42-48 (1998)). However, improved tests for cross sectional incidence testing are needed as current testing paradigms may not accurately account for variables such as ART, CD4 count, and chronic patient populations (Sakarovitch et al, J Acquir Immune Defic Syndr 45:115-122 (2007), Marinda et al, J Acquir Immune Defic Syndr 53:496-499 (2010), Hallett et al, PLoS One 4:e5720 (2009), Barnighausen et al, Epidemiology 21:685-697 (2010), McDougal et al, AIDS Res Hum Retroviruses 22:945-952 (2006), Wang and Lagakos, Biometrics (2009), Wang and Lagakos, Biometrics 66(3):864-874 (2010)). Evaluation of the timing and concentrations of p55 Gag, gp41 Env, and p66 RT IgG3 antibodies as reported here are some of the immune measurements that could be evaluated for the purpose of improving incidence testing in global populations.

### EXAMPLE 3

The concentrations of IgA antibodies in plasma and mucosal sites are shown in Table 9:

TABLE 9

| | Concentration at Peak | | | | Fold-decrease | |
|---|---|---|---|---|---|---|
| | (μg/ml for plasma) | | | | at 150 days | |
| | (μg/mg for mucosal) | | Half-life (days) | | post-peak | |
| | Est | 95% Cl | Est: | 95% Cl | Est. | 95% Cl |
| Plasma | 4.57 | 2.33, | 48.19 | 34.57, | 8.65 | 3.38, 13.93 |
| n = 12 | | 6.80 | | 61.81 | | |
| Mucosal | 8.66 | −0.03, | 2.71 | 2.06, | 6.20 | −0.51, 12.92 |
| n = 11 | | 17.36 | | 3.36 | | |

All documents and other information sources cited herein are hereby incorporated in their entirety by reference.

What is claimed is:

1. A method of determining whether a subject is recently infected with HIV-1 or is chronically infected comprising:
i) obtaining a biological sample from said subject,
ii) determining the HIV-1 specific antibody IgG3 subclass concentrations in said sample for gp41 Env, p66 RT and p55Gag, and
iii) comparing the concentrations determined in step ii) with ranges determined from recent and chronic infection to determine if the biological specimen/sample obtained from said subject has IgG3 concentrations that fall within the window of recent or chronic infection.

2. The method according to claim 1 wherein said subject is a human subject.

3. The method according to claim 1 wherein said sample is a plasma sample, serum sample or mucosal fluid.

4. The method according to claim 1 wherein said concentrations are determined using an antibody binding assay.

5. A method of determining whether a subject is recently infected with HIV-1 or is chronically infected comprising:
i) obtaining a biological sample from said subject,
ii) determining the concentration of gp41 specific IgA antibodies in said sample, and
iii) comparing the concentration determined in step ii) with ranges determined from recent and chronic infection to determine if the biological sample obtained from said subject has an gp41 specific IgA concentration that falls within the window of recent or chronic infection.

6. The method according to claim 5 wherein said subject is a human subject.

7. The method according to claim 5 wherein said sample is a plasma sample, serum sample or mucosal fluid.

8. The method according to claim 5 wherein said concentration is determined using an antibody binding assay.
